(19) United States
(12) Patent Application Publication
(10) Pub. No.: US 2026/0035345 A1
(43) Pub. Date: Feb. 5, 2026

(54) N-PIVALOYLCYSTEINE DERIVATIVES FOR THE TREATMENT OF PRESBYOPIA

(71) Applicant: Lento Bio, Inc., Potsdam, NY (US)

(72) Inventor: Kristopher BARNES, Potsdam, NY (US)

(73) Assignee: Lento Bio, Inc., Potsdam, NY (US)

(21) Appl. No.: 18/998,214

(22) PCT Filed: May 23, 2024
(86) PCT No.: PCT/US2024/030691

§ 371 Date: Jan. 24, 2025

Related U.S. Application Data
(60) Provisional application No. 63/468,674, filed on May 24, 2023.

(51) Int. Cl. C07C 323/59; A61K 9/00

(52) U.S. Cl. CPC C07C 323/59; A61K 9/0048

(57) ABSTRACT

The present invention is based on the discovery that N-pivaloylcysteine, and its derivatives, function to improve lens flexibility and treat presbyopia. N-pivaloylcysteine and N-(2-fluoro-2-methyl-1-oxopropyl)-cysteine showed improvement relative to N-acetylcysteine in reducing lens stiffness and in aqueous humor penetration. The invention includes N-pivaloylcysteine and its derivatives, their pharmaceutical compositions, and methods of administering them for the prevention of ophthalmic conditions related to lens stiffness, including presbyopia.

### TECHNICAL FIELD

The present invention relates to the therapeutic use, most notably for treating or preventing presbyopia or cataract, of N-pivaloyl-L-cysteine derivatives.

### BACKGROUND OF THE INVENTION

Presbyopia, or age-related farsightedness, is an age-related disorder resulting in improper focusing of light on the retina, which results in the progressive loss of the eye's ability to focus on nearby objects and causes issues associated with near-vision loss such as difficulty reading. Presbyopia typically manifests between the ages of 40 and 50 and is a consequence of various physiological changes in the eye, including ciliary muscle atrophy and stiffening of the lens and lens capsule. These changes together result in a loss of accommodation in the lens, leading to the symptoms of presbyopia, though stiffening of the lens has been estimated to be the strongest contributor. Lens stiffness results from damage to and cross linking of long-lived crystallin proteins, damaging the lens at a structural level and contributing to the formation of protein aggregates which may inhibit accommodation.

Cataract disease physiology also shares many characteristics with that of presbyopia which are associated with age-related crystallin protein damage. Whereas presbyopia is an earlier manifestation of lens aging, cataract formation is an older and effectively terminal manifestation. For example, both disorders have been shown to be influenced by glutathione depletion, and the presence of protective thiol compounds is important to maintain healthy lens physiology in each case.

The standard of care for presbyopia has traditionally involved corrective lenses including bifocals, progressive lenses, multifocal contacts, and the like. While the standard of care can be an effective treatment strategy, such treatments can be cumbersome, fail to restore accommodation and thus do not restore focus to the full field of view, and are temporary in nature in that they are only effective when worn. Miotic agents have been developed which restore near-vision through inducing a “pinhole effect,” but their effect is also temporary in nature and often cause side effects such as headaches. Notable miotic agents include pilocarpine (currently the only approved eyedrop for presbyopia) and aceclidine. Agents which directly reduce lens stiffness and thus restore accommodation would likely be more durable due to their mechanism of action and would avoid side effects associated with other miotics.

Miotics (including all eyedrops undergoing current clinical development) must be administered every few hours. Further, contact lenses are designed to be disposable, which leads to high costs of care. While glasses resolve this issue by being more durable, both contact lenses and glasses provide relief from symptoms only while they are being worn. Miotics can reduce depth of vision and corrective lenses do not restore accommodation and generally localize near vision to a specific section in the field of view. Monovision laser surgery, which essentially restores near vision in one eye through altering corneal refraction at the expense of distance vision, can make vision significantly less stereoscopic (and comes with other surgery-related complications and costs).

Only one current treatment, intra-ocular lens (IOL) implants, generates a lasting solution for lens damage. However, this surgical option is avoided until/unless cataracts develop. All current presbyopia-specific treatment options are merely compensatory, with no improvement in lens physiology over time. Biochemically reducing existing crosslinks would be the only technique that improves lens physiology toward its natural, healthy state.

Lipoic acid choline ester (LACE) has been shown to improve lens flexibility by reducing disulfides. LACE showed initial efficacy in a Phase 1/2 trial and was being developed for presbyopia. However, development was discontinued following the failure of LACE to meet its clinical endpoint. While LACE insufficiently restored near vision, a next-generation lens softener targeting multiple forms of damage simultaneously could overcome the shortcomings of LACE. Ursodeoxycholic acid has been compared directly to LACE. The need for optimized treatments for presbyopia remains.

N-acetylcysteine (NAC) may simultaneously treat multiple contributors of lens damage. For example, NAC functions as a disulfide breaker, AGE-inhibitor, antioxidant, metal chelator, and is the rate-limiting precursor to glutathione (GSH) synthesis which also acts via these mechanisms.

S,N-diacetylcysteine, a derivative of NAC modified at the sulfhydryl group, reduces lens stiffness in ex vivo treated aged mouse lenses through disulfide reduction as well as acetylation of lysine side chains which reduced protein aggregation and AGE formation.

NAC derivatives have been shown to increase tissue bioavailability and efficacy however acetyl group modifications have been underutilized in general and not explored in presbyopia.

### SUMMARY OF THE INVENTION

It is an objective of the present invention to provide a durable strategy for preventing and treating presbyopia, a ubiquitous disorder originating in middle-age without notable disease modifying treatment strategies, via lens flexibility enhancement.

It is an object of the present invention to provide a pharmaceutically acceptable ophthalmic composition comprising an N-pivaloyl-L-cysteine derivative of the present invention. The chemical names N-(2,2-dimethyl-1-oxopropyl)-L-cysteine, N-pivaloyl-L-cysteine and N-pivaloylcysteine are used interchangeably herein.

It is an objective of the present invention to provide NAC derivatives rationally designed to be efficacious, long-acting, and safe treatment options for presbyopia through reducing lens stiffness. The present invention is based, at least in part, on the discovery that N-pivaloylcysteine, and its derivatives, function to improve lens uptake and treat presbyopia.

The present inventors have discovered that NAC and its derivatives reduce lens stiffness, and furthermore, that N-pivaloylcysteine and its derivatives show significant improvement in effect over NAC.

The compounds of the invention have superior durability, with therapeutic effects expected to last for extended periods post-treatment and, thus, could be taken less frequently than current miotics.

The compounds of the invention also address the shortcomings of LACE which led to its failure in late-stage clinical development. Lipoic acid functions as a disulfide breaker but has not been shown to have benefits against AGEs or on GSH levels. NAC derivatives have potential multimodal effects, including reducing disulfides, and inhibiting AGEs.

The compounds of the invention will be administered as an eyedrop and improve total lens functionality and accommodative ability and restore the full field of view unlike other treatment options like miotics and corrective lenses.

This disclosure notes the following specific aspects of the invention.

N-pivaloylcysteine derivatives for any therapeutic use, including halogen-substituted derivatives, more specifically including fluorine derivatives, and pharmaceutically acceptable salts thereof.

Halogen-substituted, N-pivaloylcysteine derivatives comprising additional modifications to the C-terminus or side chain of the cysteine molecule, and pharmaceutically acceptable salts thereof.

N-pivaloylcysteine derivatives as described herein for preventing or treating presbyopia and related disorders.

A method for preventing or treating an ophthalmological disease, disorder or condition characterized by a decrease in lens flexibility and/or elasticity, comprising contacting the eye of a subject, including a human, with a N-pivaloylcysteine derivative as described herein.

A method for preventing or treating an ophthalmological disease, disorder or condition characterized by a decrease in lens flexibility, comprising, contacting the eye of a subject, including a human, with a N-pivaloylcysteine derivative as described herein, wherein the eye disease is accompanied by a decrease in accommodative function of the eye.

A method for preventing or treating presbyopia, comprising contacting the eye of a subject, including a human, with a N-pivaloylcysteine derivative as described herein.

A method for preventing or treating an ophthalmological disease, disorder, or condition, comprising, contacting the eye of a subject, including a human, with a N-pivaloylcysteine derivative as described herein, wherein the chosen route of administration is via an ophthalmic route.

A composition, comprising a N-pivaloylcysteine derivative as described herein, formulated as an eye drop, or onto a contact lens, or as an eye cream or gel.

A composition, comprising a N-pivaloylcysteine derivative as described herein, wherein the concentration of N-pivaloylcysteine, or a derivative thereof, or a pharmaceutically acceptable salt thereof comprised in the formulation is 0.000005 to 15% (w/v).

Any and or all aspects of the invention described above may be separately selected and/or combined.

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The articles “a” and “an” refer to one or to more than one (i.e., to at least one) of the grammatical object of the article unless the context clearly indicates otherwise. By way of example, “an element” means one element or more than one element.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

The present methods, compounds, and systems are not limited to specific methods, compounds, components, or compositions described or exemplified herein. It is also to be understood that the terminology used herein is for the purpose of describing and is not intended to be limiting.

Novel compositions of matter are described for therapeutic purposes, as active ingredients in the treatment for disease in humans. These agents may be used to restore flexibility to the eye lens and/or to restore accommodation to vision.

One aspect of the invention is N-pivaloylcysteine derivatives of the following formula:

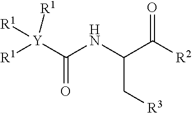

wherein
each R¹ is independently optionally substituted C₁₋₆ alkyl, C₁₋₆ aryl, halogen, a halogen-containing group, or an ammonium group;
Y is —C— or —CO—, or —N—;
R² is OR⁴, SR⁴, SeR⁴, or N(R⁴)₂ and
R³ is SR⁴, SeR⁴, or SC(O)—R⁴;
   where R⁴ is hydrogen or optionally substituted C₁₋₆ alkyl.

Another aspect of the present invention is N-pivaloylcysteine derivatives of the following formula:

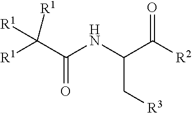

wherein
each R¹ is independently optionally substituted C₁₋₆ alkyl, C₁₋₆ aryl, halogen, a halogen-containing group, or an ammonium group;
R² is OR⁴, SR⁴, SeR⁴, or N(R⁴)₂ and
R³ is SR⁴, SeR⁴, or SC(O)—R⁴;
   where R⁴ is hydrogen or optionally substituted C₁₋₆ alkyl.

Optional substituents may be selected from alkyl, cycloalkyl, aryl, heteroaryl, hydroxy, alkoxy, halogen, and the like without limitation, as well as conjugates and functional groups, solubilizing groups, lipid groups, and the like for optimizing pharmacokinetic and other properties.

As used herein, unless otherwise noted, “alkyl” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g., C₁₋₆) shall refer independently to the number of carbon atoms in an alkyl moiety or to the alkyl portion of a larger alkyl-containing substituent. Non-limiting examples of alkyl groups include methyl, ethyl, n-propyl, cyclopropyl, iso-propyl, n-butyl, sec-butyl, iso-butyl, tert-butyl, and the like.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C₁₋₁₀ amine group, a nitro group (—NO₂), a C₁₋₁₀ alkyl group, a C₃₋₁₀ cycloalkyl group, a C₆₋₁₂ aryl group, a C₁₋₁₀ alkoxy group, a C₁₋₁₀ trifluoroalkyl group such as a trifluoromethyl group (—CF₃) and the like, or a cyano group (—CN). Exemplary substituents include alkyl, alkylidenyl, alkylcarboxy, alkoxy, alkenyl, alkenylcarboxy, alkenyloxy, aryl, aryloxy, alkylaryl, alkylaryloxy, —OH, amide, carboxamide, carboxy, sulfonyl, ═O, ═S, —NO₂, halogen, haloalkyl, fused saturated or unsaturated optionally substituted rings, —S(O)R, —SO₃R, —SR, —NRR′, —OH, —CN, —C(O)R, —OC(O)R, —NHC(O)R, —(CH₂)ₙCO₂R or —(CH₂)ₙCONRR′ where n is 0-4, and wherein R and R′ are independently H, alkyl, aryl or alkylaryl. Substituents also include replacement of a carbon atom and one or more associated hydrogen atoms.

As used herein, “bioisosteric group” and “bioisostere,” are used interchangeably to mean chemical substituents to functional groups meant to have roughly similar chemical or physical properties and to produce generally similar biological effects.

A “halogen-containing group” includes, but is not limited to, haloalkyl groups, such as halomethyl, dihalomethyl, trihalomethyl, preferably fluoromethyl, chloromethyl, bromomethyl, iodomethyl, difluoromethyl, trifluoromethyl, dichloromethyl, trichloromethyl, dibromomethyl, tribromomethyl, diiodomethyl, triiodomethyl, fluoroethyl, fluoropropyl, fluoroisopropyl, fluoroisobutyl, and fluoro(tert)butyl.

In one embodiment of the invention, at least one R¹ is halogen, preferably fluorine. In another embodiment of the invention, one or more of the R¹ groups is fluorine, preferably one of the R¹ groups is fluorine and the other R¹ groups are C₁₋₆ alkyl. In another embodiment of the invention, one or more of R¹ is a halogen. In another embodiment of the invention, one or more of R¹ is a halogen containing group. In another embodiment of the invention, one of the R¹ groups is halogen or a halogen-containing group and the other R¹ groups are methyl (CH₃). In another embodiment of the invention, each R¹ group is a halogen or optionally substituted C₁₋₆ alkyl, and the compound is used to treat presbyopia or another ophthalmological disorder.

In another embodiment of the invention, R² is OH, as in the case of cysteine derivatives. In another embodiment of the invention R² is OR⁴, where R⁴ is optionally substituted C₁₋₆ alkyl. In another embodiment of the invention, R² is N(R⁴)₂. In another embodiment of the invention, R² is SR⁴ or SeR⁴. In another embodiment of the invention, R² as well as the adjacent carbonyl are replaced with a bioisosteric group, such as an imidazole, oxazole, thiazole, or selenazole, any salt thereof, and the like.

In one embodiment of the invention, R³ is SH, as in the case of cysteine derivatives. In another embodiment of the invention, R³ is SeH, as is the case with selenocysteine derivatives. In another embodiment of the invention, R³ is a selenoether, SeR⁴, where R⁴ is optionally substituted C₁₋₆ alkyl.

A preferred compound of the invention is N-(2-fluoro-2-methyl-1-oxopropyl)-cysteine, pictured below.

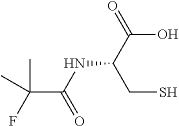

### Specific Benefits of Invention

Compounds of the invention alleviate the underlying causes of presbyopia with multiple advantages over standard of care treatments, including the following compounds:

Derivatives of N-acetylcysteine include, but are not limited to, N-acetophenyl-cysteine, N-cyclopropylcarbonyl-cysteine, N-(2,2-dimethyl-1-oxopropyl)-cysteine (“N-pivaloylcysteine”), N-(3-methyl-2-butanyl)-cysteine, N-(2-ethyl-2-methyl-1-oxopropyl)-cysteine, N-(2-phenyl-2-methyl-1-oxopropyl)-cysteine, N-(2-methoxy-2-methyl-1-oxopropyl)-cysteine, N-(2-ammonium-2-methyl-1-oxopropyl)-cysteine, and N-(2-fluoro-2-methyl-1-oxopropyl)-cysteine).

Furthermore, two derivatives, N-pivaloylcysteine and N-(2-fluoro-2-methyl-1-oxopropyl)-cysteine, showed improvement relative to N-acetylcysteine.

In the embodiments described herein, an N-pivaloylcysteine derivative of the invention may be used in a composition in ranges of 1% to 10% w/v, 1% to 5% w/v, 1% to 2% w/v, 1% to 1.5% w/v, more preferably above 1% w/v and below 1.5% w/v, for example 1.16% w/v to 1.32% w/v, or 1.1875% w/v to 1.3125% w/v. Additional ranges of N-pivaloylcysteine derivatives that may be used include 0.95% w/v to 1.2% w/v, 1.1% w/v to 1.4% w/v, and 1.2% w/v to 1.3% w/v. A preferred amount of N-pivaloylcysteine derivative is 1.25% w/v. Other amounts of N-pivaloylcysteine derivative that may be used include for example and without limitation, 0.5% w/v, 0.6% w/v, 0.7% w/v, 0.8% w/v, 0.9% w/v, 0.95% w/v, 0.99% w/v, 1% w/v, 1.01% w/v, 1.05% w/v, 1.08% w/v, 1.1% w/v, 1.15% w/v, 1.2% w/v, 1.21% w/v, 1.22% w/v, 1.23% w/v, 1.24% w/v, 1.26% w/v, 1.27% w/v, 1.28% w/v, 1.29% w/v, 1.3% w/v, 1.31% w/v, 1.32% w/v, 1.35% w/v, 1.4% w/v, 1.45% w/v, 1.49% w/v, and 1.5% w/v, and ranges and amounts between any of these selected amounts of N-pivaloylcysteine derivative.

For treating of presbyopia according to the invention, the N-pivaloylcysteine derivatives, or their pharmaceutically acceptable salts, analogs, metabolites, prodrugs, derivatives, metabolites, co-crystals, modifications, solvates, hydrates, isotopes, tautomers, esters, polymorphs, or stereoisomers, can be administered in combination with one or more other medications, including but not limited to, miotic agents such as pilocarpine.

In certain embodiments, the method further comprises administering to the eye of the patient an agent that facilitates reduction of the patient's pupil or improvement in visual performance.

In certain embodiments, the additional agent is administered to said eye of the patient concurrently with the dosage of N-pivaloylcysteine derivative. In certain embodiments, the additional agent is administered to said eye of the patient sequentially either before or after administering to said eye of the patient the dosage of N-pivaloylcysteine derivative.

The method may be further characterized according to the identity of the additional agent. In certain embodiments, the additional agent improves visual performance. In certain embodiments, the additional agent facilitates reduction of the patient's pupil. In certain embodiments, the additional agent is selected from the group consisting of a muscarinic acetylcholine receptor agonist, an alpha-2 adrenergic agonist, a prostaglandin, and a miotic agent.

The pharmaceutically acceptable ophthalmic composition may comprise an N-pivaloylcysteine derivative of the present invention as the sole active ingredient in the pharmaceutically acceptable ophthalmic composition. In some embodiments, the pharmaceutically acceptable ophthalmic composition further comprises boric acid, sodium citrate dihydrate, sodium chloride, hydrochloric acid and/or sodium hydroxide, and water.

The pharmaceutically acceptable ophthalmic composition may be administered once daily. The pharmaceutically acceptable ophthalmic composition may be administered twice daily.

The pharmaceutically acceptable ophthalmic composition may be administered to both eyes of the patient. The pharmaceutically acceptable ophthalmic composition may be administered to one eye of the patient. The pharmaceutically acceptable ophthalmic composition may be administered to the nondominant eye of the patient. The pharmaceutically acceptable ophthalmic composition may be administered to the dominant eye of the patient.

Additional pharmaceutically acceptable ingredients may be formulated together with compounds of the invention, particularly in the form of an eyedrop. Examples of these additives may include tonicity modulators, buffers, surfactants, thickeners, corneal penetration enhancers, stabilizers, preservatives, antioxidants, solubilizers, suspending agents, pH adjusters, excipients, binders, fluidizers, lubricants, solvents, and the like.

Tonicity modulators may include nonionic compounds, and ionic agents including salts such as NaCl and the like.

Surfactants include ionic surfactants (including anionic surfactants and cationic surfactants) as well as nonionic surfactants.

Thickeners may include hydroxypropyl methylcellulose and the like.

Corneal penetration enhancers may include detergents, particularly benzalkonium chloride (BAK), EDTA, and the like

Buffers may include sodium dihydrogenphosphate, sodium hydrogen phosphate, other derivatives of phosphoric acid or salts thereof, and the like.

Solubilizers may include Tween-80, Polyethylene Glycol, cyclodextrins including hydroxypropyl-beta cyclodextrin as well as other alpha and beta cyclodextrins, and the like.

The compositions described herein may comprise a suitable preservative. Examples of suitable preservatives include benzalkonium chloride (“BAK”), Polyquaternium-1 (Polyquad®), chlorobutanol, stabilized chlorine dioxide, and others. Stabilized chlorine dioxide, also known as Purite®, may be described as an aqueous solution of sodium chlorite (NaClO₂). U.S. Pat. No. 5,424,078, which is incorporated herein by reference in its entirety, further discusses the use of stabilized chlorine dioxide as a preservative for ophthalmic formulations.

Above mentioned additive ingredients including tonicity modulators, buffers, surfactants, thickeners, corneal penetration enhancers, stabilizers, preservatives, antioxidants, solubilizers, suspending agents, pH adjusters, excipients, binders, fluidizers, lubricants, solvents, and the like may be utilized at any relevant and pharmaceutically acceptable concentration.

Solvents may include water, saline and the like.

In addition to eye drops, the invention includes compositions comprising a N-pivaloylcysteine derivative as described herein, formulated onto a contact lens, or as an eye cream or gel. limited to chitosan, hyaluronic acid, poloxamer, PLGA, PVCL-PVA-PEG, cetalkonium chloride, glean gum, hydroxyethylcellulose, or gelatin.

Another aspect of the invention provides a method of treating presbyopia in a patient according to a monotherapy treatment regimen, comprising administering to an eye of a patient in need thereof a dosage of a single therapeutic agent in an amount effective for treatment of presbyopia, wherein the single therapeutic agent is an N-pivaloylcysteine derivative of the invention antagonist.

The method may be further characterized by additional features, such as the dosing regimen and the identity of the dosage, as described in more detail below. The invention embraces all permutations and combinations of these features. Accordingly, in certain embodiments, the dosage is administered at or near the bedtime of the patient. In certain embodiments, the dosage is administered within 1 hour of the patient's bedtime.

In certain embodiments, the dosage is administered as a single eye drop. In certain embodiments, the dosage is administered as two or more eye drops.

In the embodiments described herein, compositions may be administered once daily, twice daily, or more. Preferably, the compositions are administered once daily.

When administered, the compositions preferably have a duration of action sufficient for an entire day. The duration of action refers to the duration of time that the administered composition has an effect on at least one vision parameter or ocular condition (e.g., presbyopia). In some embodiments, the compositions may have a duration of effect of at least two hours, at least three hours, preferably at least four hours, more preferably at least six hours, more preferably at least eight hours, even more preferably at least 10 hours, as well as all intervening time points. Some embodiments may provide for a composition having a duration of action greater than hours, for example 12 hours, or even 24 hours.

In certain embodiments, the method provides a therapeutic effect against presbyopia for a duration of at least 12 hours. In certain embodiments, the method provides a therapeutic effect against presbyopia for a duration of at least 16 hours. In certain embodiments, the method provides a therapeutic effect against presbyopia for a duration of at least 18 hours. In certain embodiments, the method provides a therapeutic effect against presbyopia for a duration of at least 20 hours. In certain embodiments, the method provides a therapeutic effect against presbyopia for a duration of at least 24 hours. In certain embodiments, the method provides a therapeutic effect against presbyopia for a duration of at least 36 hours. In certain embodiments, the method provides a therapeutic effect against presbyopia for a duration of at least 48 hours.

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

### EXAMPLES

### Example 1. Synthesis

Unless otherwise noted, reactions were conducted in oven-dried glassware (140° C.) under an atmosphere of nitrogen gas (N₂) using anhydrous solvents. S-trityl L-cysteine methyl ester was prepared starting from commercially available L-cysteine methyl ester hydrochloride according to a known procedure (Gale, et al., Inorg. Chem., 50: 10460-10471 (2011). The general reaction scheme is listed below, followed by specific information for each step.

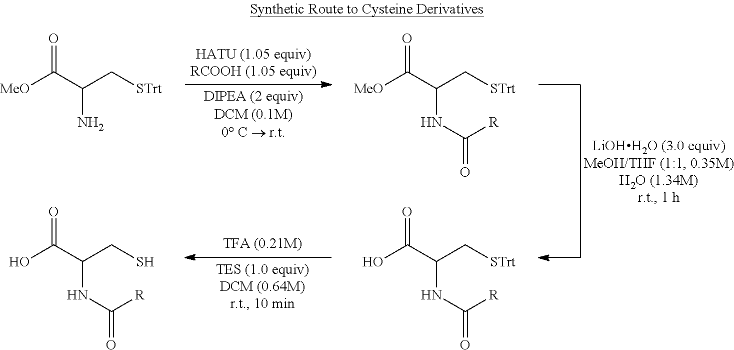

N-2,2-dimethyl-propionyl S-trityl L-cysteine methyl ester: A mixture of S-trityl L-cysteine methyl ester (1.00 g, 2.64 mmol), hexafluorophosphate azabenzotriazole tetramethyl uronium (HATU, 1.05 g, 2.77 mmol), and pivalic acid (283 mg, 2.77 mmol) in dichloromethane (DCM) (26.4 mL) was cooled to 0° C. To this solution, DIPEA (0.92 mL, 5.28 mmol) was added dropwise. After full addition, the mixture was allowed to stir for 10 min at 0° C. before bringing to room temperature. After 1 hour, the reaction mixture was transferred to a separatory funnel and washed with NaHCO₃ (2×20 mL) followed by water (2×20 mL), and brine (1×10 mL). The organic layer was dried with MgSO₄, filtered, and concentrated under reduced pressure. The resulting crude residue was purified by flash chromatography (SiO₂, 3:17 EtOAc/hexanes) to afford the title compound as an off-white solid (1.15 g, 2.49 mmol, 94%). ¹H NMR (600 MHz, CDCl₃) δ 7.38 (dt, J=8.5, 1.8 Hz, 6H), 7.28 (t, 6H), 7.22 (t, 3H), 6.24 (d, J=7.6 Hz, 1H), 4.59 (ddd, J=7.6, 5.6, 4.6 Hz, 1H), 3.71 (s, 3H), 2.64 (dd, J=12.1, 4.7 Hz, 1H), 2.58 (dd, J=12.1, 5.7 Hz, 1H), 1.21 (s, 9H); ¹³C NMR (150 MHz, CDCl₃) δ 178.2, 171.3, 144.4, 129.6, 128.1, 127.0, 66.8, 52.7, 51.1, 38.9, 34.1, 27.5.

N-2,2-dimethyl-propionyl S-trityl L-cysteine: To a solution of N-2,2-dimethyl-propionyl S-trityl L-cysteine methyl ester (1.14 g, 2.47 mmol) in 1:1 THF/MeOH (7.06 mL), water (1.84 mL) was added followed by LiOH·H₂O (311 mg, 7.41 mmol). After 1 hour of stirring, the reaction mixture was cooled to 0° C., adjusted to a pH of 2 using 1.0 M HCl, and transferred to a separatory funnel. The aqueous layer was extracted with DCM (3×25 mL). The combined organic extracts were washed with water (1×25 mL) and brine (1×25 mL); and dried with MgSO₄, filtered, and concentrated under reduced pressure to provide the title compound. The resulting solid was used without further purification (1.04 g, 2.32 mmol, 94%). ¹H NMR (600 MHz, CDCl₃) δ 7.41 (d, 6H), 7.29 (t, J=7.7 Hz, 6H), 7.23 (t, 3H), 6.16 (d, J=6.5 Hz, 1H), 4.22 (td, J=6.8, 4.8 Hz, 1H), 2.72 (qd, J=12.9, 5.9 Hz, 2H), 1.20 (s, 9H); ¹³C NMR (151 MHz, CDCl3) δ 180.2, 172.9, 144.3, 129.6, 128.3, 127.1, 67.2, 51.8, 38.9, 32.9, 27.5.

N-2,2-dimethyl-propionyl L-cysteine: To mixture of N-2,2-dimethyl-propionyl S-trityl L-cysteine (211 mg, 0.47 mmol) in DCM (0.73 mL), TFA (2.24 mL) and triethylsilane (0.07 mL, 0.47 mmol) were added sequentially. After 10 min of stirring, the reaction mixture was concentrated under reduced pressure. The resulting crude solid was purified by flash chromatography (SiO₂, 3:100 i-PrOH/DCM) to afford the title compound as a white solid (81 mg, 0.40 mmol, 84%). ¹H NMR (400 MHz, CDCl3) δ 6.67 (d, J=6.8 Hz, 1H), 4.82 (dt, J=6.9, 4.3 Hz, 1H), 3.15 (ddd, 1H), 2.99 (ddd, J=14.3, 9.6, 4.6 Hz, 1H), 1.46 (t, 1H), 1.26 (s, 9H); ¹³C NMR (151 MHz, CDCl3) δ 179.7, 173.3, 53.5, 39.1, 27.5, 26.5.

N-2-methyl-2-fluoro-propionyl S-trityl L-cysteine methyl ester: Following the procedure from N-2,2-dimethyl-propionyl S-trityl L-cysteine methyl ester, the title compound was prepared from S-trityl L-cysteine methyl ester (3.28 g, 6.68 mmol), HATU (2.67 g, 7.01 mmol), 2-fluoroisobutyric acid (0.66 mL, 7.01 mmol), and DIPEA (2.33 mL, 13.4 mmol) in DCM (67 mL). Purification by flash chromatography (SiO₂, 15:100 EtOAc/hexanes) afforded the title compound (2.39 g, 5.13 mmol, 77%) as pale-yellow solid. ¹H NMR (400 MHz, CDCl₃) δ 7.39 (d, 6H), 7.29 (t, J=7.5 Hz, 6H), 7.23 (t, 3H), 6.92-6.84 (m, 1H), 4.52 (q, J=6.3 Hz, 1H), 3.71 (s, 3H), 2.70-2.57 (m, 2H), 1.56 (dd, 6H); ¹³C NMR (151 MHz, CDCl₃) δ 170.6, 144.4, 129.7, 128.2, 127.1, 67.1, 52.9, 50.9, 33.9, 25.2, 25.1, 24.9; ¹⁹F NMR (376 MHz, CDCl₃) δ-147.51.

N-2-methyl-2-fluoro-propionyl S-trityl L-cysteine: Following the procedure from N-2,2-dimethyl-propionyl S-trityl L-cysteine, the title compound was prepared from N-2-methyl-2-fluoro-propionyl S-trityl L-cysteine methyl ester (2.39 g, 5.13 mmol), LiOH·H₂O (646 mg, 15.4 mmol), and water (3.83 mL) in 1:1 THF/MeOH (14.7 mL). No further purification was necessary to afford the title compound as a pale-yellow solid (2.13 g, 4.72 mmol, 92%). ¹H NMR (600 MHz, CDCl₃) δ 7.42 (d, J=7.7 Hz, 6H), 7.31 (t, J=7.7 Hz, 6H), 7.25 (t, J=7.2 Hz, 3H), 6.87 (t, J=6.6 Hz, 1H), 4.39 (d, J=6.6 Hz, 1H), 2.74 (qd, J=12.8, 5.7 Hz, 2H), 1.69-1.46 (dd, 6H); ¹³C NMR (151 MHz, CDCl₃) δ 173.7, 173.5, 144.1, 129.5, 128.1, 127.0, 96.6, 95.4, 67.2, 50.9, 33.1, 25.0, 24.9, 24.8, 24.8; ¹⁹F NMR (376 MHz, CDCl₃) δ-147.39.

N-2-methyl-2-fluoro-propionyl L-cysteine: Following the procedure from N-2,2-dimethyl-propionyl L-cysteine, the title compound was prepared from N-2-fluoro-2-methyl-propionyl S-trityl L-cysteine (250 mg, 0.56 mmol), TFA (2.64 mL), and triethylsilane (0.06 mL, 0.56 mmol) in DCM (0.87 mL). The resulting crude solid was dissolved in water (10 mL) and Et₂O (10 mL) and transferred to a separatory funnel. The aqueous layer was washed with Et₂O (3×10 mL) and lyophilized to afford the title compound as a white solid (104 mg, 0.50 mmol, 89%). ¹H NMR (600 MHz, CDCl₃) δ 7.23 (t, 1H), 4.91-4.85 (m, 1H), 3.14 (ddd, J=14.2, 8.7, 4.2 Hz, 1H), 3.03 (ddd, J=14.2, 9.4, 4.6 Hz, 1H), 1.67-1.53 (dd, 6H), 1.48 (t, J=9.0 Hz, 1H); ¹³C NMR (151 MHz, CDCl₃) δ 174.1, 173.9, 173.4, 96.7, 95.5, 53.3, 26.5, 25.2, 25.1, 24.9, 24.8; ¹⁹F NMR (376 MHz, CDCl3) δ-147.48.

### Example 2. Ex Vivo Lense Stiffness

An ex vivo screen was conducted to evaluate the effect of multiple compounds on lens stiffness using a more efficient variant of the coverslip method (Cheng, et al., J. Vis. Exp., 111: 53986 (2016). Our variant uses a single measurement point to enable multidrug screening, utilizing a weight deemed optimal based on coverslip quantity-response curves. Previously dissected and frozen eyes from 8-month-old C57BL/6 mice were thawed, and the ocular lenses were removed via micro-dissection. Next, intact ocular lenses were immersed and incubated in test compound solutions at various concentrations for 12 hours at 37° C. Individual washed lenses were placed into a divot in a solution-filled plexiglass measurement chamber, where uncompressed axial diameter was assessed. Then 3 glass coverslips glued together, with a total weight of 588.0 mg, were placed on top of the lens, and the axial diameter was remeasured. Change in lens stiffness was calculated as the ratio between the axial diameter of compressed lens to the axial diameter of the unweighted lens (compressed/uncompressed), with higher ratios indicative of more flexibility.

NAC (20 mM) significantly improved lens stiffness compared to untreated (vehicle) control lenses in the screen.

To address an important limitation with ex vivo lens treatment (that lens stiffness increases over time, by default, when the lens is removed from the eye post-mortem), we compared treatment with NAC to freshly dissected 8-month lenses and observed natural stiffening occurred during the 12-hour incubation period (10.34% mean compression, n=10). Thus, to account for the possibility that NAC was primarily acting to slow default stiffening rather than breaking pre-existing crosslinks, we tested stiffness after 1-hour of incubation with NAC and found a significant improvement of 2.07% compared to vehicle.

### Example 3

An initial in vivo test was conducted on NAC utilizing 7-month-old C57BL/6 mice subsequently treated for one month 2×/day with both 100 mM and 20 mM NAC eyedrops (artificial tears was used as the vehicle). During the last day, animals were sacrificed, eyes enucleated, and lenses were removed. Lens stiffness was measured by the same assay as ex vivo, with measurements taken within 5-10 minutes of lens removal. Both a 588 mg and 1047 mg weight were used in the assay. NAC 100 mM showed significant improvement in lens stiffness over vehicle with both coverslip weights, and NAC 20 mM showed minor and insignificant improvement. A later study showed that N-acetyl-D-cysteine, the enantiomer of NAC, also shows significant improvement versus vehicle but at a reduced power versus NAC.

All compounds were tested at 20 mM concentrations, and if solubility in media was insufficient to achieve this concentration, 0.1% tween-80 and 0.4% PEG-400 were added to the vehicle. At first, several acetyl derivatives modified at the R² site (N-acetophenyl-cysteine, N-acetobenzyl-cysteine, N-cyclopropylcarbonyl-cysteine, N—(N,N-dimethylformamidyl-cysteine, N-(2,2-dimethyl-1-oxopropyl)-cysteine (“N-pivaloylcysteine”), and N-pentanyl-cysteine) were generated, of which N-pivaloylcysteine showed the largest improvement. Subsequently, multiple other N-pivaloyl variants with modifications to the R¹ site (N-(3-methyl-2-butanyl)-cysteine, N-(2-ethyl-2-methyl-1-oxopropyl)-cysteine, N-(2-phenyl-2-methyl-1-oxopropyl)-cysteine, N-(2-methoxy-2-methyl-1-oxopropyl)-cysteine, N-(2-ammonium-2-methyl-1-oxopropyl)-cysteine, and N-(2-fluoro-2-methyl-1-oxopropyl)-cysteine were tested ex vivo. Two compounds, N-pivaloylcysteine and N-(2-fluoro-2-methyl-1-oxopropyl)-cysteine, were shown to have significant improvement over NAC, and several others showed improvement over control and insignificant improvement over NAC (Table 1). N-(2-fluoro-2-methyl-1-oxopropyl)-cysteine had the highest mean significance as well as the highest significance vs vehicle.

TABLE 1

| | | Avg. % | T-Test vs |
|---|---|---|---|
| Compound Name | Image | Compress. | Vehicle |
| Vehicle* | | 4.35 | NA |
| N-acetyl-cysteine | 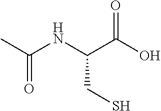 | 5.76 | 1.46E−04 |
| N-phenylcarbonyl-cysteine | 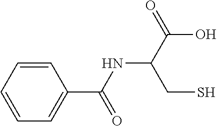 [SMILES: O=C(NC(CS)C(=O)O)c1ccccc1] | 5.98 | 0.0074 |
| N-benzylcarbonyl-cysteine | 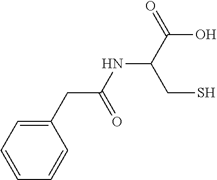 [SMILES: O=C(Cc1ccccc1)NC(CS)C(=O)O] | 4.38 | 0.9453 |
| N-cyclopropylcarbonyl- cysteine | 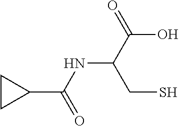 [SMILES: O=C(NC(CS)C(=O)O)C1CC1] | 6.02 | 0.0052 |
| N-dimethylaminocarbonyl- cysteine | 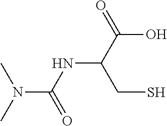 | 4.19 | ND |
| N-(2,2-dimethyl-1- oxopropyl)-cysteine (“N- pivaloylcysteine”) | 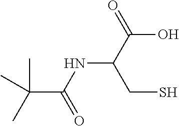 | 6.82 | 3.15E−08 |
| N-(n-pentylcarbonyl)- cysteine | 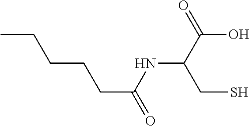 | 4.31 | ND |
| N-(isopropylcarbonyl)- cysteine | 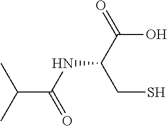 | 6.71 | 8.12E−04 |
| N-(2-ethyl-2-methyl-1- propylcarbonyl)-cysteine | 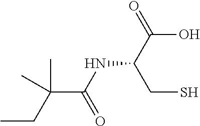 | 6.29 | 2.43E−04 |
| N-(2-phenyl-2-methyl-1- propylcarbonyl)-cysteine | 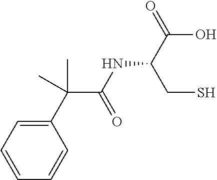 [SMILES: CC(C)(C(=O)N[C@@H](CS)C(=O)O)c1ccccc1] | 6.18 | 2.39E-05 |
| N-(2-methoxy-2-methyl-1- propylcarbonyl)-cysteine | 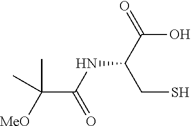 | 6.38 | 2.39E-05 |
| N-(2-ammonium-2-methyl-1- propylcarbonyl)-cysteine | 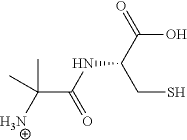 | 5.68 | 0.0239 |
| N-(2-fluoro-2-methyl-1- propylcarbonyl)-cysteine | 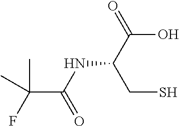 | 6.87 | 3.37E−08 |

*Vehicle is a combination of Epilife (60 μM CaCl) treated lenses and Epilife + 0.1% Tween-80 and 0.4% PEG-400, aggregated here as no significant or no worthy difference was observed between the two. Phenyl-, benzyl-, and cyclobutene- substituted variants used Epilife; all others used Epilife + PEG/Tween as vehicle to enhance solubility of the test agent.

### Example 4. In Vivo Efficacy

Rationale and goal: To determine the effect of N-acetylcysteine (NAC) and its variants on reducing lenses stiffness in vivo. Certain variables differ significantly between in vivo and ex vivo studies, most notably the presence of the cornea as a barrier during in vivo treatment, the 12-hour duration of ex vivo vs 28-day duration of in vivo treatment, and the effect of post-mortem lens stiffening ex vivo.

The following sample groups were tested in 7-month-old C57BL/6 mice: vehicle (artificial tears)(40 mice), N-acetylcysteine 20 mM (20 mice), N-(2-fluoro-2-methyl-1-propylcarbonyl)-cysteine 200 mM (10 mice), LACE (lipoic acid choline ester) 1.5% (PBS vehicle) (10 mice), 2-month-old mice (untreated) (7 mice).

Lens stiffness was significantly reduced in mice treated with N-(2-fluoro-2-methyl-1-propylcarbonyl)-cysteine versus both LACE and vehicle treated mice. Vehicle-treated mice averaged 199.34 micrometers total compression (std. dev 32.16). Mice treated with N-(2-fluoro-2-methyl-1-propylcarbonyl)-cysteine had an average of 281.05 micrometers total compression (std. dev 40.70). Mice treated with LACE averaged 209.68 micrometers total compression (std. dev 28.16). The two-month-old young comparators had an average of 319.71 micrometers total compression (std. dev 40.05). N-(2-fluoro-2-methyl-1-propylcarbonyl)-cysteine was superior to 8-month vehicle (p<0.0001) and LACE (p<0.0001) and is able to restore 67.9% of lens stiffness lost to aging from 2-8 months. N-(2-fluoro-2-methyl-1-propylcarbonyl)-cysteine also showed a significant improvement over NAC. LACE itself did not significantly improve upon vehicle treated lenses.

### Example 5. Toxicology and Dosing Optimization in Mice

A study was conducted to assess the maximum tolerable dose of N-(2-fluoro-2-methyl-1-propylcarbonyl)-cysteine (and its S-acetylated counterpart, N-(2-fluoro-2-methyl-1-propylcarbonyl)-S-acetylcysteine for lens stiffness reduction and to assess via histology whether any safety issues arose from 1 week of treatment with these compounds, particularly in the lens and cornea.

Two-month-old C57BL/6 mice were treated 2×/day with treatment compounds (see sample groups (n=5) and concentrations below). Animals were assessed for itching and scratching/blinking after dosing, hyperemia, lid swelling, discharge, and squinting, all on a scale of 1-4. Those scoring were blinded to treatment groups. Following euthanasia eyes were fixed and stained with H+E, and corneal and lens anatomy in the two highest concentration groups considered tolerable were assessed.

| | Dose | Dose |
|---|---|---|
| Group | Concentration | Volume |
| Vehicle (artificial tears) | N/A | |
| N-(2-fluoro-2-methyl-1- | 200 mM | 3 μL/eye |
| propylcarbonyl)-S-acetylcysteine | | |
| N-(2-fluoro-2-methyl-1- | 300 mM | |
| propylcarbonyl)-S-acetylcysteine | | |
| N-(2-fluoro-2-methyl-1- | 200 mM | |
| propylcarbonyl)-cysteine | | |
| N-(2-fluoro-2-methyl-1- | 100 mM | |
| propylcarbonyl)-cysteine | | |
| N-(2-fluoro-2-methyl-1- | 100 mM | |
| propylcarbonyl)-cysteine | | |

N-(2-fluoro-2-methyl-1-propylcarbonyl)-cysteine and its S-acetylated counterpart N-(2-fluoro-2-methyl-1-propylcarbonyl)-S-acetylcysteine had a maximum soluble concentration in artificial tears of 200 mM and 300 mM, respectively. Blink rate post-dosing was increased in all treatment groups, though it returned to normal pre-dosing. Hyperemia mildly increased at 200 mM N-(2-fluoro-2-methyl-1-propylcarbonyl)-cysteine, while squinting, lid swelling and discharged were unchanged. Histology was assessed to be entirely normal in N-(2-fluoro-2-methyl-1-propylcarbonyl)-cysteine treated lenses. N-(2-fluoro-2-methyl-1-propylcarbonyl)-cysteine at 200 mM was assessed to be tolerable.

### Example 6. Aqueous Humor LC/MS Concentration Comparison

A study was conducted to assess the degree to which N-(2-fluoro-2-methyl-1-propylcarbonyl)-cysteine penetrated the mouse cornea, as compared to NAC and N-pivaloylcysteine.

Eight-month-old C57BL/6 mice were given 3 μM eyedrops of 100 mM N-(2-fluoro-2-methyl-1-propylcarbonyl)-cysteine, 100 mM N-pivaloylcysteine, or 100 mM NAC for 1 month at 2×/day. 10 mice were present in each group. No noticeable increase of tolerability issues including blinking or hyperemia were observed over time. Safety via histology was positive.

On the final day aqueous humor was drawn from the eyes (average of 1-5 μL extracted) 1 hour after final dose, and the concentration of each compound quantified via LC/MS (column: Atlantis T3, 100×2.1 mm, 5 μm; mobile phases: A: Water/Formic Acid (1000/5, v/v), B: Acetonitrile/Methanol/Formic Acid (50/50/0.3, v/v/v).

N-(2-fluoro-2-methyl-1-propylcarbonyl)-cysteine showed the highest aqueous humor concentration (47 μM), followed by N-pivaloylcysteine (38 μM). NAC was much lower, with 17 out of 20 samples falling below the limit of detection.

N-(2-fluoro-2-methyl-1-propylcarbonyl)-cysteine demonstrated improved aqueous humor concentration over NAC and N-pivaloylcysteine and thus improved corneal penetration. N-(2-fluoro-2-methyl-1-propylcarbonyl)-cysteine also demonstrated an additional advantage of superior lens stiffness reducing efficacy ex vivo where there is no cornea, potentially by increasing lens penetration as well.

1.-28. (canceled)

29. A compound of the formula: 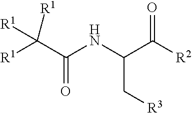
wherein
each R¹ is independently optionally substituted C₁₋₆ alkyl, C₁₋₆ aryl, halogen, a halogen-containing group, or an ammonium group;
R² is OR⁴, SR⁴, SeR⁴, or N(R⁴)₂;
R³ is SR⁴, SeR⁴, or SC(O)—R⁴, where R⁴ is hydrogen or optionally substituted C₁₋₆ alkyl, and pharmaceutically acceptable salts thereof; and
wherein one or more of the R¹ substituents is a halogen or halogen-containing group.

30. A compound according to claim 29, wherein R³ is SR⁴.

31. A compound according to claim 30, wherein R² is OR⁴.

32. A compound according to claim 31, wherein one or more of the R¹ substituents is fluorine.

33. A compound according to claim 32, wherein R⁴ is hydrogen.

34. The compound N-(2-fluoro-2-methyl-1-oxopropyl)-cysteine of the formula: 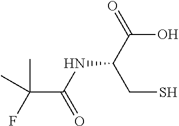
or a pharmaceutically-acceptable salt or ester thereof.

35. A pharmaceutically acceptable ophthalmic composition comprising a compound according to claim 1 and one or more pharmaceutically acceptable excipients selected from the group consisting of tonicity modulators, buffers, surfactants, thickeners, corneal penetration enhancers, stabilizers, preservatives, antioxidants, solubilizers, suspending agents, pH adjusters, excipients, binders, fluidizers, lubricants, and solvents.

36. An ophthalmic composition according to claim 35, formulated as an eyedrop.

37. A method of reducing eye lens stiffness, comprising administering to a subject in need thereof, a therapeutically effective amount of a compound of the formula: 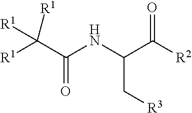
wherein
each R¹ is independently optionally substituted C₁₋₆ alkyl, C₁₋₆ aryl, halogen, a halogen-containing group, or an ammonium group;
R² is OR⁴, SR⁴, SeR⁴, or N(R⁴)₂ and
R³ is SR⁴, SeR⁴, or SC(O)—R⁴, where R⁴ is hydrogen or optionally substituted C₁₋₆ alkyl, and pharmaceutically acceptable salts thereof.

38. A method according to claim 37, wherein R² is OR⁴ and R³ is SR⁴.

39. A method according to claim 38, wherein one or more of the R¹ substituents is a halogen or halogen-containing group.

40. A method according to claim 39, wherein one or more of the R¹ substituents is fluorine.

41. A method according to claim 40, wherein R⁴ is hydrogen.

42. A method according to claim 37, wherein said compound is the compound N-(2-fluoro-2-methyl-1-oxopropyl)-cysteine of the formula: 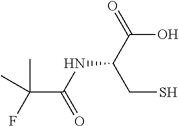
or a pharmaceutically-acceptable salt or ester thereof.

43. A method of treating presbyopia, comprising administering to a subject in need thereof, a therapeutically effective amount of a compound of the formula: 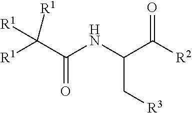
wherein
each R¹ is independently optionally substituted C₁₋₆ alkyl, C₁₋₆ aryl, halogen, a halogen-containing group, or an ammonium group;
R² is OR⁴, SR⁴, SeR⁴, or N(R⁴)₂ and
R³ is SR⁴, SeR⁴, or SC(O)—R⁴, where R⁴ is hydrogen or optionally substituted C₁₋₆ alkyl, and pharmaceutically acceptable salts thereof.

44. A method according to claim 43, wherein R² is OR⁴ and R³ is SR⁴.

45. A method according to claim 44, wherein one or more of the R¹ substituents is a halogen or halogen-containing group

46. A method according to claim 45, wherein one or more of the R¹ substituents is fluorine.

47. A method according to claim 46, wherein R⁴ is hydrogen.

48. A method according to claim 43, wherein said compound is the compound N-(2-fluoro-2-methyl-1-oxopropyl)-cysteine of the formula: 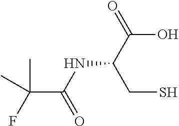
or a pharmaceutically-acceptable salt or ester thereof.
